(19) United States
(12) Patent Application Publication
(10) Pub. No.: US 2013/0204120 A1
(43) Pub. Date: Aug. 8, 2013

(54) EQUIPMENT AND METHODOLOGIES FOR MAGNETICALLY-ASSISTED DELIVERY OF THERAPEUTIC AGENTS THROUGH BARRIERS

(71) Applicants: WEINBERG MEDICAL PHYSICS LLC, Bethesda, MD (US); Irving N. Weinberg, Bethesda, MD (US)

(72) Inventor: Irving N. WEINBERG, Bethesda, MD (US)

(73) Assignees: WEINBERG MEDICAL PHYSICS LLC, Bethesda, MD (US); Irving N. Weinberg, Bethesda, MD (US)

(21) Appl. No.: 13/761,200

(22) Filed: Feb. 7, 2013

Related U.S. Application Data
(60) Provisional application No. 61/596,395, filed on Feb. 8, 2012.

(51) Int. Cl. A61N 2/00; A61B 5/01; A61N 2/02; A61B 5/055

(52) U.S. Cl. CPC A61N 2/004; A61B 5/055; A61B 5/015; A61N 2/02
USPC 600/411; 600/12

(57) ABSTRACT

Magnetic gradients are used to transport Magnetic Nano Particles through a barrier, for example, the cribiform (also spelled “cribriform”) plate, which is a porous bony structure which separates the nasal cavity from the cranial vault. By utilizing a configuration of magnets (whether of the electromagnetic type or permanent magnets), MNPs can be propelled, pushed, pulled or otherwise manipulated in relation to an anatomical and/or physiological barrier, to position, re-position or maintain the position(s) of the MNPs.

### CROSS-REFERENCE TO RELATED APPLICATION(S)

This application relies for priority on United States Provisional Patent Application Ser. No. 61/596,395, entitled “MAGNETICALLY ASSISTED DELIVERY OF THERAPEUTIC AGENTS THROUGH BARRIERS,” filed on Feb. 8, 2012, the entirety of which being incorporated by reference herein.

### FIELD OF THE INVENTION

Disclosed embodiments are directed, generally, to facilitate transportation of therapeutic agents across an anatomic and/or physiological barrier.

### DESCRIPTION OF THE RELATED ART

Many scholarly works have been published concerning means for delivering therapeutic agents across an anatomic or physiological barrier. A classical approach to this endeavor is to coat the therapeutic agent with a material that is naturally transported across cells making up the barrier.

### SUMMARY

However, conventional transportation of therapeutic agents across barriers is accomplished without the benefit of applied magnetic fields. The present invention contemplates the use of Magnetic Nano-Particles (MNPs) with or without additional coatings to aid in transport across barriers.

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Disclosed embodiments use magnetic gradients to transport MNPs through a barrier, for example, the cribiform (also spelled “cribriform”) plate, which is a porous bony structure which separates the nasal cavity from the cranial vault. By utilizing a configuration of magnets (whether of the electromagnetic type or permanent magnets), MNPs can be propelled, pushed, pulled or otherwise manipulated in relation to an anatomical and/or physiological barrier, to position, re-position or maintain the position(s) of the MNPs.

An additional aspect is the use of imaging to guide the transport.

An additional aspect is the insertion of an applicator into a body orifice, wherein the applicator may contain a magnet for propelling MNPs across the barrier.

### BRIEF DESCRIPTION OF THE DRAWINGS

A more compete understanding of the present invention and the utility thereof may be acquired by referring to the following description in consideration of the accompanying drawings, in which like reference numbers indicate like features, and wherein:

FIGS. 1(a)-(b) are an image set obtained with MRI of the front part of a rat brain in which magnetic nano-particles have been deposited in the right nostril, prior to and after application of a magnetic gradient

FIG. 2 illustrates disclosed embodiments directed to transporting magnetic nano-particles across the cribiform plate

### DETAILED DESCRIPTION

The description of specific embodiments is not intended to be limiting of the present invention. To the contrary, those skilled in the art should appreciate that there are numerous variations and equivalents that may be employed without departing from the scope of the present invention. Those equivalents and variations are intended to be encompassed by the present invention.

In the following description of various invention embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Moreover, it should be understood that various connections are set forth between elements in the following description; however, these connections in general, and, unless otherwise specified, may be either direct or indirect, either permanent or transitory, and either dedicated or shared, and that this specification is not intended to be limiting in this respect.

In accordance with at least one disclosed embodiment, one or more magnetic gradients are used to transport Magnetic Nano Particles (MNPs) through an organic barrier, for example, the cribiform plate, which is a porous bony structure which separates the nasal cavity from the cranial vault.

For the purposes of this disclosure, the term “organic barrier” is intended to convey an anatomic, physiologic, or combined anatomic-physiologic barrier. It is understood that a physiologic barrier can be composed of microscopic anatomic barriers (for example, tight junctions in vessel walls).

In accordance with at least one disclosed embodiment, a configuration of magnetic elements (e.g., electromagnetic or permanent magnetic material) is provided, configured and utilized to push (i.e., repel) or pull (i.e., attract) MNPs across the organic barrier.

In accordance with at least one disclosed embodiment, imaging is used to guide transport of the MNPs across the organic barrier.

In accordance with at least one disclosed embodiment, an applicator is provided and inserted into a body orifice, wherein the applicator may contain a magnet to assist in transporting MNPs across the organic barrier.

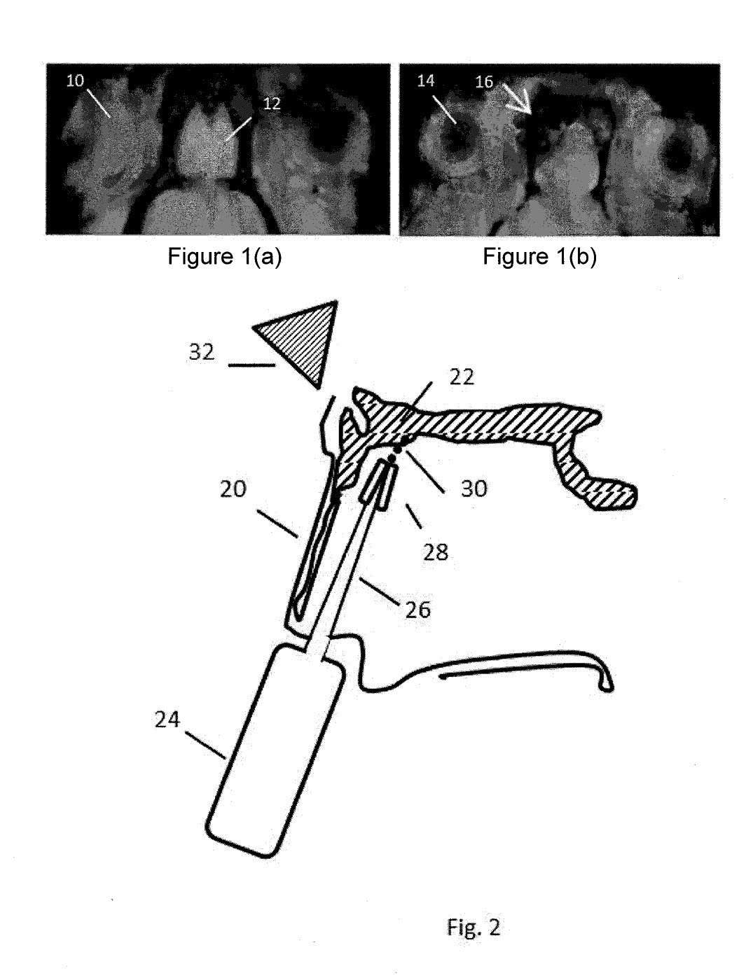

FIGS. 1(a)-(b) are images obtained using Magnetic Resonance Imaging (MRI) technology. The image is of the front part of a rat brain 100 in which MNPs have been deposited in the right nostril 105, prior to and after application of a magnetic gradient.

FIGS. 1(a)-(b) provide a set of magnetic resonance images of a rat to which MNPs had been administered intra-nasally (on one side of the nose). FIG. 1(a) shows the rat before application of a magnetic gradient. As shown in FIG. 1(a), the structure indicated by item 110 is the olfactory bulb of the rat brain 100. As shown in FIG. 1(b), the rat brain 100 after application of the magnetic gradient.

As indicated in FIG. 1(b), the arrow 115 refers to a reduced MRI signal in the portion of the olfactory bulb 110 on the same side as the intranasal administration 105, indicating the new presence of MNPs (which have distorted the local magnetic field so that the MRI signal from nearby protons has been reduced). This demonstrates and evidences that MNPs had been transported across the cribiform plate 115 into the rat's brain 100. Accordingly, FIGS. 1(a)-(b) confirms the potential use of MRI to describe the spatial distribution of the MNPs.

FIG. 2 provides an illustration used to explain the operation and design of various embodiments directed at transportation of MNPs across an organic barrier, e.g., the cribiform plate. As shown in FIG. 2, the sagittal slice of the nasal cavity 200 also shows the cribiform plate at a superior location 205.

In accordance with at least one disclosed embodiment, an applicator 210 provided as a mechanism for conveying MNPs 215 (for example, in solution or as a powder) to the inside of the nasal cavity 200, may be attached to a nozzle or tube 220. Along or near the nozzle or tube 220 may be affixed a magnet or array of magnetic elements 225, which can be configured to assist in transportation, via either repelling or attracting, of the MNPs 215 across the cribiform plate 205.

Alternatively, the magnet or array of magnetic elements 225 may be replaced or augmented by another magnet or array of magnetic elements 230 provided externally to the nasal cavity. As a result, this secondary magnet or array of magnetic elements may be used to assist in propulsion of the MNPs 215, for example, by pulling or pushing the MNPs across the cribiform plate.

Although the disclosed embodiments have been described in conjunction in relationship to the introduction of MNPs via a subject's nasal cavity, it should be understood that the use of the nasal cavity is merely an example of an orifice that can be entered with an applicator. Thus, other body cavities or orifices could be similarly used to introduce MNPs and/or magnets for transport of MNPs. As a result, the configuration, size and shape of an applicator and corresponding and including magnets or magnetic elements would differ according to the particular configuration of the orifice.

Thus, by utilizing a configuration of magnets (whether of the electromagnetic type or permanent magnets), MNPs can be propelled, pushed, pulled or otherwise manipulated in relation to an anatomical and/or physiological barrier, to position, re-position or maintain the position(s) of the MNPs. This configuration of magnets may be utilized to create a local magnetic field minimum at a location distal to the barrier (as disclosed in patent publication WO2010099552, entitled “DEVICES, SYSTEMS AND METHODS FOR MAGNETIC-ASSISTED THERAPEUTIC AGENT DELIVERY,” the disclosure of which being incorporated by reference in its entirety).

It should also be appreciated that disclosed embodiment may be used to introduce MNPs into various parts of a subject's body including at the cellular level. It is known that MNPs may be resident in living cells, for example, stem cells. Accordingly, in accordance with at least one disclosed embodiment, the methodologies for introducing and transporting MNPs can be used to deliver therapy by repelling or attracting (e.g., pushing or pulling) MNP-laden cells to manipulate or effect the placement of such cells in a subject's body.

Thus, at least one disclosed embodiment may be utilized to effect transportation of cells, e.g., for stem cell treatments, wherein MNP laden stem cells may be transported by the disclosed embodiment into damaged tissue in order to treat disease or injury. As a result of transportation of those stem cells into the damaged tissue, the stem cells self-renew and give rise to subsequent generations with variable degrees of differentiation capacities wherein new tissue may be generated and replace diseased and damaged areas in the subject's body, with minimal risk of rejection and side effects.

Likewise, it should be understood that the disclosed embodiments are not limited to implementation with only stem cell therapies. Rather, disclosed embodiments may be utilized with any number of cellular therapies wherein particular cells are introduced into diseased or damages areas of a subject's body for the purpose of treating disease or repairing tissue.

Furthermore, disclosed embodiments may be utilized in connection with and to effect or support bone marrow transplantation. In fact, any medical treatment procedure that requires or would benefit from the transportation of therapeutic substances across a physiological or anatomic barrier could be implemented, effected or further supported by the disclosed methods for transporting the MNPs and associated materials, e.g., cellular material, medicine, etc. Thus, although the above description refers to an example wherein the organic barrier is a cribiform plate and the ultimate goal is to affect transportation of the MNPs into the central nervous system, it should be understood that this is merely an illustrative example. The cribiform plate is a porous bony structure that separates the nasal cavity from the cranial vault. The present invention is not limited to this barrier site.

In summary, disclosed embodiments may be provided to effect transportation of MNPs (and potentially also associated therapeutic agents) using one or more magnetic gradients. Transportation of such MNPs across organic, e.g., physiological and anatomic barriers may, therefore, be effected. Other aspects of the present invention should be apparent to those skilled in the art based on the discussion provided herein.

Thus, disclosed embodiments provide an apparatus and method of augmenting or implementing transport across an anatomic or physiological barrier, with the use of MNPs, which are affected by magnetic gradients applied in the vicinity of the barrier. These MNPs may be associated with a therapeutic agent, or may be used themselves for therapy. It is known that MNPs can be attracted to magnets (i.e., pulled), or repelled by magnets (i.e., pushed). Accordingly, disclosed embodiments use one or more magnetic arrays that can push and/or pull MNPs. Thus, disclosed embodiments may be used to deliver drugs, cells, diagnostic agents, and other materials to organs or structures in the central nervous system or in other body compartments of a subject.

Thus, therapeutic agents may be attached to, or encapsulated within, an MNP. In accordance with at least one disclosed embodiment, the MNPs have magnetic cores with a polymer or metal coating. Alternatively the therapeutic agent may be coated with a porous polymer that contains magnetic nano-particles precipitated within the pores.

Once transported across an organic barrier and delivered to a site, the MNPs may be heated through the application of an oscillating magnetic field (i.e., hyperthermia).

The progress of MNPs in a body may be accomplished with MRI as in FIG. 1, and alternatively through the use of magnetic particle imaging.

By introducing the MNP/therapeutic agent combination into a subject's bloodstream the MNP/therapeutic agent combination may then be subsequently transported and positioned within the subject's body via manipulation with magnets and/or an array of magnetic elements that produce magnetic fields. These magnets or magnetic elements may be provided within or externally to the subject's body.

While this invention has been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

For example, the disclosed embodiments may be used to implement, facilitate, enhance, or improve the efficacy of gene therapy protocols, e.g., the use of DNA as a pharmaceutical agent to treat disease by using the DNA to supplement or alter genes within an individual's cells as a therapy to treat disease or condition, and/or cell therapy protocols, e.g., wherein new cells are introduced into a tissue in order to treat a disease or condition, for example, via stem cell treatments, cell-based gene therapy, xenotransplantation, transplantation of transdifferentiated cells, etc.

Additionally, it should be understood that the functionality described in connection with various described components of various invention embodiments may be combined or separated from one another in such a way that the architecture of the invention is somewhat different than what is expressly disclosed herein. Moreover, it should be understood that, unless otherwise specified, there is no essential requirement that methodology operations be performed in the illustrated order; therefore, one of ordinary skill in the art would recognize that some operations may be performed in one or more alternative order and/or simultaneously.

Various components of the invention may be provided in alternative combinations operated by, under the control of or on the behalf of various different entities or individuals.

Further, it should be understood that, in accordance with at least one embodiment of the invention, system components may be implemented together or separately and there may be one or more of any or all of the disclosed system components. Further, system components may be either dedicated systems or such functionality may be implemented as virtual systems implemented on general purpose equipment via software implementations.

As a result, it will be apparent for those skilled in the art that the illustrative embodiments described are only examples and that various modifications can be made within the scope of the invention as defined in the appended claims.

What is claimed is:

1. An apparatus comprising:
one or more magnets positioned and utilized to augment transport of magnetizable particles across a barrier within a subject.

2. The apparatus of claim 1, wherein a drug is bound to at least one of the magnetizable particles during at least one portion of the augmented transport process.

3. The apparatus of claim 1, where at least one of the magnetizable particles is less than one-micron in maximal diameter.

4. The apparatus of claim 1, wherein at least one of the magnetizable particles is less than 1000 nanometers in maximal diameter.

5. The apparatus of claim 1, wherein at least one of the magnetizable particles is less than 100 nanometers in maximal diameter.

6. The apparatus of claim 1, wherein the barrier contains living cells.

7. The apparatus of claim 1, wherein at least one of the magnetizable particles has super-paramagnetic properties.

8. The apparatus of claim 1, where the configuration of magnets creates a local magnetic field minimum at a location distal to the barrier.

9. The apparatus of claim 1, where at least one of the magnets is inserted into a body orifice of the subject.

10. The apparatus of claim 1, where at least one of the magnets is an electromagnetic.

11. The apparatus of claim 1, where the barrier is the blood-brain barrier.

12. The apparatus of claim 1, where the barrier is the cribiform plate of the subject.

13. The apparatus of claim 1, where the particles are tracked using magnetic resonance imaging.

14. The apparatus of claim 1, where the particles are tracked using magnetic particle imaging.

15. A method of augmenting transport of magnetizable particles across a barrier by utilizing a configuration of one or more magnets, the method comprising:
positioning the one or more magnets in proximity to the magnetizable particles; and
repelling or attracting the magnetizable particles using the at least one magnet to augment transport of magnetizable particles across a barrier within a subject.

16. The method of claim 13, wherein a drug is bound to at least one of the magnetizable particles during at least one portion of the repelling or attracting.

17. The method of claim 13, where at least one of the magnetizable particles is less than one-micron in maximal diameter.

18. The method of claim 13, wherein at least one of the magnetizable particles is less than 1000 nanometers in maximal diameter.

19. The method of claim 13, wherein at least one of the magnetizable particles is less than 100 nanometers in maximal diameter.

20. The method of claim 13, wherein the barrier contains living cells.

21. The method of claim 13, wherein at least one of the magnetizable particles has super-paramagnetic properties.

22. The method of claim 13, where the configuration of magnets creates a local magnetic field minimum at a location distal to the barrier.

23. The method of claim 13, where at least one of the magnets is inserted into a body orifice of the subject.

24. The method of claim 13, where at least one of the magnets is an electromagnetic.

25. The method of claim 13, where the barrier is the blood-brain barrier.

26. The method of claim 13, where the barrier is the cribiform plate of the subject.

27. The apparatus of claim 1, wherein the particles are tracked using magnetic resonance imaging.

28. The apparatus of claim 1, wherein the particles are tracked using magnetic particle imaging.

29. The method of claim 13, further comprising tracking the particles using magnetic resonance imaging.

30. The method of claim 13, further comprising tracking the particles using magnetic particle imaging.
